(19) United States
(12) Patent Application Publication
(10) Pub. No.: US 2026/0284667 A1
(43) Pub. Date: Sep. 24, 2026

(54) A METHOD AND SYSTEM FOR AMPLIFICATION OF NUCLEIC ACIDS USING POLYMERASE CHAIN REACTION (PCR)

(71) Applicant: Merck Patent GmbH, Darmstadt (DE)

(72) Inventors: Camille Correia, Bensheim (DE); Kai Jaehrling, Lautertal (DE); Philipp Lau, Darmstadt (DE); Ludger Beylage, Reinheim (DE)

(21) Appl. No.: 19/163,898

(22) PCT Filed: Mar. 19, 2024
(86) PCT No.: PCT/EP2024/057301

§ 371 Date: Sep. 10, 2025

(30) Foreign Application Priority Data
Mar. 20, 2023 (EP) 23162961.9

(51) Int. Cl. B01L 7/00; C12Q 1/686

(52) U.S. Cl. CPC B01L 7/5255; C12Q 1/686; B01L 2300/0838; B01L 2300/0883; B01L 2300/185; B01L 2400/0487

(57) ABSTRACT

The disclosure herein relates to a system for large-scale production of nucleic acids using polymerase chain reaction (PCR) and methods of use thereof.

### RELEVANT FIELD

Embodiments described herein relate to a method and system for large-scale production of nucleic acids using polymerase chain reaction (PCR). More specifically, some embodiments relate to a method using a robotic arm to transfer a reaction vessel comprising capillary tubing between PCR steps.

### BACKGROUND

Polymerase chain reaction (PCR) is a method used in molecular biology for the amplification of nucleic acids in vitro. The method relies on an initial activation step and repeated cycles of three additional steps: 1) denaturation of double-stranded deoxyribonucleic DNA into two accessible single strands, 2) annealing of complementary primers to each of the single strands, and 3) elongation, also referred to as extension, of nucleic acid chains using a polymerase enzyme to synthesize a new complementary DNA strand. Conventional PCR is performed in plates with multiple 20-250 μL conical vials, which are placed in a thermal cycling PCR instrument, also referred to as a thermocycler, comprising a thermal heating block.

Using microchannels based on microfluidic capillary or chip designs as a reaction vessel for amplification of DNA provides a number of advantages, such as rapid heat transfers, compact design, high-throughput capacity, minimal footprints, and ease of integrating automation. Microfluidic-based PCR instruments include spiral and oscillating flow designs. (See, Kopp et al. 1998 Micro Total Anal. Syst. 98 7-10, which is hereby incorporated by reference in its entirety)

Chip-based designs have limited working volume and recently have been surpassed in popularity by spiral capillary designs incorporating either a metal heating block (Peltier elements) or baths containing thermal media as heat exchangers. (See, KR20050078568; US20080145923; Park et al., 2003 Anal. Chem. 75 6029-33; and Kim et al. 2016 Bull. Korean Chem. Soc. 37 1878-81, each of which is hereby incorporated by reference in its entirety) The heating element may be divided in multiple zones of varying sizes and temperature corresponding to the optimum conditions for the activation, denaturation, annealing, and elongation steps. DNA is amplified as a continuous process by feeding a reaction mixture through a capillary tube wound around the heating elements. This design suffers from the same disadvantages of a microchannel chip, namely lack of flexibility at an affordable price. In order to produce a wide portfolio of DNA targets with differing protocols, a numbering up strategy, as well as access to multiple chips or heating element designs would be necessary, resulting in increased production costs. (See, U.S. Pat. No. 8,163,489, which is hereby incorporated by reference in its entirety) Alternatively, a multi-segmented heating block may be used. For these devices, some variability in PCR protocols is tolerated by temperature control of each section/segment, which allows for limited modification of reaction times by controlling the number of segments used. The physical scalability of these designs is restricted by the low contact area and heat transfers between an electric heating element and a capillary tube. The use of a bath with thermal media does not suffer this constraint as the outer tube is fully immersed in a medium.

A fully continuous PCR device employing four temperature-controlled fluid chambers and capillary tubing through which the master mix is pumped has been previously described. (See, U.S. Pat. No. 7,217,699, which is hereby incorporated by reference in its entirety) To maximize yield, multiple cycles are performed by the solution exiting the final chamber reenters the first chamber and continuing along the microchannel path.

Another version of a PCR device includes multiple designs for nucleic acid amplification, one of which being the pumping of a reaction mixture in a continuous and repetitive loop between two temperature-controlled fluid baths. (See, U.S. Pat. No. 5,720,923, which is hereby incorporated by reference in its entirety)

Scalability remains the greatest limitation to industrial production of nucleic acids using PCR. Large volume production is typically performed through a “numbering-up” approach by increasing the number of thermocycling reactions in microliter plates and combining the products. On the other hand, a physical size increase, often referred to as “sizing up”, using milliliter or liter scale conical tubes, or similar biocontainers may be used to perform amplification. “Numbering up” takes advantage of the high surface-area-volume-ratio in microliter conical tubes for rapid heat transfers. However, there are several disadvantages, such as greater waste generated as well as larger footprint and increased cost due to additional devices required for filling and emptying of the microliter tubes. In comparison, the approach of physical “sizing up” uses milliliter or liter scale conical tubes, or similar biocontainers often result in low yields and inconsistent quality due to inefficient mixing and inability to regulate temperature control in such large vessels.

Capillary bioreactors take advantage of the rapid heat and mass transfers on small dimensions while circumventing the redundancy of “numbering-up”. As described above, these devices commonly rely on microliter platforms and examples of scale up are limited. (See, U.S. Pat. No. 8,163,489, which is incorporated by reference in its entirety) Moreover, the present versions of capillary bioreactors cannot be easily adapted to a wide range of reaction times without significant effort to re-design the surface area of heating element. Further, larger DNA targets require additional time per elongation cycle for the later cycles often in PCR protocols. This would be impossible to perform in a continuous reactor as described above because the flow rate, capillary size and capillary length dictate the reaction time, which cannot be adjusted during a run.

For commercial manufacturing, it is imperative that the production device can be very easily modified or programmed to accommodate a wide range of reaction conditions supplying a large portfolio of PCR products.

A system to amplify a variety of nucleic acids targets in large volumes, specifically a method resulting in a high quality product and a high level of reproducibility, represents an inventive advance in the art.

### SUMMARY

The shortcomings of the prior art are overcome by embodiments described herein.

Some embodiments include a system for performing polymerase chain reaction (PCR) comprising: a reaction vessel comprising capillary tubing wound around a frame; a circulation device for circulating a reaction mixture through the reaction vessel; at least two temperature-controlled baths holding a fluid for thermal transfer to the reaction mixture; and at least one manipulator to transfer the reaction vessel among the temperature-controlled baths while performing PCR.

In some embodiments, the manipulator is a multi-axis pulley or a robotic arm. In some embodiments, the manipulator is a robotic arm. In some embodiments, the robotic arm is automated. In some embodiments, the robotic arm is programmable. In some embodiments, the robotic arm is manual. In some embodiments, the robotic arm comprises rotary joints. In some embodiments, the circulation device is a pump. In some embodiments, the pump is at least one type of pump selected from the group consisting of: a peristaltic pump, a gear pump, a lobe pump, a membrane pump, and a syringe pump. In some embodiments, the system is a discrete batch bioreactor. In some embodiments, the capillary tubing is wound around the frame in a design selected from the designs consisting of: simple helical coils, 180 degree turns, and other more complex coiled flow inverters (CFI). In some embodiments, the capillary tubing comprises at least one material selected from the group consisting of: metals, plastics, and silicones. In some embodiments, the capillary tubing is silicone tubing. In some embodiments, the capillary tubing is platinum-cured silicone tubing. In some embodiments, the capillary tubing is peroxide-cured silicone tubing. In some embodiments, the capillary tubing is sterilizable. In some embodiments, the capillary tubing is single use. In some embodiments, the circulation device creates oscillatory flow. In some embodiments, the system comprises three temperature-controlled baths.

Some embodiments include a method for performing polymerase chain reaction (PCR) using the system of claim 1 to produce a product, the method comprising: filling the reaction vessel with the reaction mixture; introducing turbulence using the circulation device to circulate the reaction mixture; submerging the reaction vessel into a first temperature-controlled bath corresponding to reaction conditions for a denaturation step of PCR; and transferring the reaction vessel using a manipulator from the first temperature-controlled bath to a second temperature-controlled bath corresponding to reaction conditions for a different step of PCR.

In some embodiments, the product is produced in a milliliter or a liter volume scale. In some embodiments, each submerging step corresponds to a step of PCR. In some embodiments, the method further comprising programming the movement of the robotic arm prior to performing PCR. In some embodiments, the product is produced in a batch size from 1 mL to 5 L. In some embodiments, the product is produced in a batch size within a range selected from the group consisting of: 1 mL to 100 mL, 50 mL to 200 mL, 150 mL to 300 mL, 250 mL to 400 mL, 350 mL to 500 mL, 450 mL to 600 mL, 550 mL to 700 mL, 650 mL to 800 mL, 750 mL to 900 mL, 850 mL to 1L, 950 mL to 1.5 L, 1 L to 2 L, 1.5 L to 3 L, 2 L to 3.5 L, 2.5 L to 4 L, 3 L to 4.5 L, and 3.5 L to 5 L. In some embodiments, the method further comprising prior to the filling step calculating the diameter and length of the capillary tubing based on the volume of a batch. In some embodiments, the method further comprising, prior to the filling step, calculating the diameter and length of the capillary tubing based on the rate of heat transfer required for PCR. In some embodiments, the method further comprising filling the reaction vessel with the reaction mixture in a segmented form. In some embodiments, the method further comprising filling the reaction vessel with the reaction mixture in a non-segmented form. In some embodiments, the method further comprising, prior to introducing turbulence, calculating the flow rate of the reaction mixture from at least one characteristic selected from the group consisting of: the optimal amount of mixing and turbulence, the dimensions of the capillary tubing, and the winding design of the capillary tubing.

### BRIEF DESCRIPTION OF THE DRAWINGS

FIG. 1 provides a diagram of the reactor capillary tubing, pumps, connections, and heated baths.

The appended drawings illustrate some embodiments of the disclosure herein and are therefore not to be considered limiting in scope, for the invention may admit to other equally effective embodiments. It is to be understood that elements and features of any embodiment may be found in other embodiments without further recitation and that, where possible, identical reference numerals have been used to indicate comparable elements that are common to the figures.

### DETAILED DESCRIPTION

The present disclosure describes some embodiments of a discrete batch, capillary-based bioreactor capable of DNA amplification using PCR on milliliter and liter volume scales. In some embodiments, the bioreactor comprises a reaction vessel of capillary tubing to be filled with reaction mixture; two or more temperature controlled baths; an automated, mechanical, manipulator such as a robotic arm; and a pump connected to the capillary tubing. In some embodiments, the reaction time for each PCR step is fully controlled by the robotic arm as the reaction mixture in the capillary tubing is submerged in a temperature-controlled bath, held, removed, and transferred to a subsequent temperature-controlled bath. Each sequence of submersion in a temperature-controlled bath, hold, and removal from a temperature-controlled bath represents one reaction step of the PCR protocol.

### I. Methods

Some embodiments herein describe a method of large-scale production of nucleic acids using polymerase chain reaction (PCR).

The time of each reaction step is determined by the optimum PCR protocol for each master mix. In some embodiments, the Activation step is performed for a time period selected from the range consisting of: 10 seconds to 10 minutes. In some embodiments, the Activation step is performed for a time period within in the range of 5 seconds to 1 minute. In some embodiments, the Activation step is performed for a time period within in the range of 1 minute to 2 minutes. In some embodiments, the Activation step is performed for a time period within in the range of 2 minutes to 3 minutes. In some embodiments, the Activation step is performed for a time period within in the range of 3 minutes to 4 minutes. In some embodiments, the Activation step is performed for a time period within in the range of 4 minutes to 5 minutes. In some embodiments, the Activation step is performed for a time period within in the range of 5 minutes to 6 minutes. In some embodiments, the Activation step is performed for a time period within in the range of 6 minutes to 7 minutes. In some embodiments, the Activation step is performed for a time period within in the range of 7 minutes to 8 minutes. In some embodiments, the Activation step is performed for a time period within in the range of 8 minutes to 9 minutes. In some embodiments, the Activation step is performed for a time period within in the range of 9 minutes to 10 minutes.

In some embodiments, the Activation step is performed for 10 seconds. In some embodiments, the Activation step is performed for 11 seconds. In some embodiments, the Activation step is performed for 12 seconds. In some embodiments, the Activation step is performed for 13 seconds. In some embodiments, the Activation step is performed for 14 seconds. In some embodiments, the Activation step is performed for 15 seconds. In some embodiments, the Activation step is performed for 16 seconds. In some embodiments, the Activation step is performed for 17 seconds. In some embodiments, the Activation step is performed for 18 seconds. In some embodiments, the Activation step is performed for 19 seconds. In some embodiments, the Activation step is performed for 20 seconds. In some embodiments, the Activation step is performed for 21 seconds. In some embodiments, the Activation step is performed for 22 seconds. In some embodiments, the Activation step is performed for 23 seconds. In some embodiments, the Activation step is performed for 24 seconds. In some embodiments, the Activation step is performed for 25 seconds. In some embodiments, the Activation step is performed for 26 seconds. In some embodiments, the Activation step is performed for 27 seconds. In some embodiments, the Activation step is performed for 28 seconds. In some embodiments, the Activation step is performed for 29 seconds. In some embodiments, the Activation step is performed for 30 seconds. In some embodiments, the Activation step is performed for 31 seconds. In some embodiments, the Activation step is performed for 32 seconds. In some embodiments, the Activation step is performed for 33 seconds. In some embodiments, the Activation step is performed for 34 seconds. In some embodiments, the Activation step is performed for 35 seconds. In some embodiments, the Activation step is performed for 36 seconds. In some embodiments, the Activation step is performed for 37 seconds. In some embodiments, the Activation step is performed for 38 seconds. In some embodiments, the Activation step is performed for 39 seconds. In some embodiments, the Activation step is performed for 40 seconds. In some embodiments, the Activation step is performed for 41 seconds. In some embodiments, the Activation step is performed for 42 seconds. In some embodiments, the Activation step is performed for 43 seconds. In some embodiments, the Activation step is performed for 44 seconds. In some embodiments, the Activation step is performed for 45 seconds. In some embodiments, the Activation step is performed for 46 seconds. In some embodiments, the Activation step is performed for 47 seconds. In some embodiments, the Activation step is performed for 48 seconds. In some embodiments, the Activation step is performed for 49 seconds. In some embodiments, the Activation step is performed for 50 seconds. In some embodiments, the Activation step is performed for 51 seconds. In some embodiments, the Activation step is performed for 52 seconds. In some embodiments, the Activation step is performed for 53 seconds. In some embodiments, the Activation step is performed for 54 seconds. In some embodiments, the Activation step is performed for 55 seconds. In some embodiments, the Activation step is performed for 56 seconds. In some embodiments, the Activation step is performed for 57 seconds. In some embodiments, the Activation step is performed for 58 seconds. In some embodiments, the Activation step is performed for 59 seconds. In some embodiments, the Activation step is performed for 1 minute. In some embodiments, the Activation step is performed for 2 minutes. In some embodiments, the Activation step is performed for 3 minutes. In some embodiments, the Activation step is performed for 4 minutes. In some embodiments, the Activation step is performed for 5 minutes. In some embodiments, the Activation step is performed for 6 minutes. In some embodiments, the Activation step is performed for 7 minutes. In some embodiments, the Activation step is performed for 8 minutes. In some embodiments, the Activation step is performed for 9 minutes. In some embodiments, the Activation step is performed for 10 minutes. In some embodiments, the Activation step is performed for less than 10 minutes. In some embodiments, the Activation step is performed for greater than 10 minutes. In some embodiments, the Activation step is performed for greater than 10 seconds.

In some embodiments, the Denaturation step is performed for a time period selected from the range consisting of: 10 seconds to 10 minutes. In some embodiments, the Denaturation step is performed for a time period within in the range of 5 seconds to 1 minute. In some embodiments, the Denaturation step is performed for a time period within in the range of 1 minute to 2 minutes. In some embodiments, the Denaturation step is performed for a time period within in the range of 2 minutes to 3 minutes. In some embodiments, the Denaturation step is performed for a time period within in the range of 3 minutes to 4 minutes. In some embodiments, the Denaturation step is performed for a time period within in the range of 4 minutes to 5 minutes. In some embodiments, the Denaturation step is performed for a time period within in the range of 5 minutes to 6 minutes. In some embodiments, the Denaturation step is performed for a time period within in the range of 6 minutes to 7 minutes. In some embodiments, the Denaturation step is performed for a time period within in the range of 7 minutes to 8 minutes. In some embodiments, the Denaturation step is performed for a time period within in the range of 8 minutes to 9 minutes. In some embodiments, the Denaturation step is performed for a time period within in the range of 9 minutes to 10 minutes.

In some embodiments, the Denaturation step is performed for 1 minute. In some embodiments, the Denaturation step is performed for less than 1 minutes. In some embodiments, the Denaturation step is performed for 2 minutes. In some embodiments, the Denaturation step is performed for 3 minutes. In some embodiments, the Denaturation step is performed for 4 minutes. In some embodiments, the Denaturation step is performed for 5 minutes. In some embodiments, the Denaturation step is performed for 6 minutes. In some embodiments, the Denaturation step is performed for 7 minutes. In some embodiments, the Denaturation step is performed for 8 minutes. In some embodiments, the Denaturation step is performed for 9 minutes. In some embodiments, the Denaturation step is performed for 10 minutes. In some embodiments, the Denaturation step is performed for less than 10 minutes. In some embodiments, the Denaturation step is performed for greater than 10 minutes. In some embodiments, the Denaturation step is performed for greater than 10 seconds.

In some embodiments, the Annealing step is performed for a time period selected from the range consisting of: 10 seconds to 10 minutes. In some embodiments, the Annealing step is performed for a time period within in the range of 5 seconds to 1 minute. In some embodiments, the Annealing step is performed for a time period within in the range of 1 minute to 2 minutes. In some embodiments, the Annealing step is performed for a time period within in the range of 2 minutes to 3 minutes. In some embodiments, the Annealing step is performed for a time period within in the range of 3 minutes to 4 minutes. In some embodiments, the Annealing step is performed for a time period within in the range of 4 minutes to 5 minutes. In some embodiments, the Annealing step is performed for a time period within in the range of 5 minutes to 6 minutes. In some embodiments, the Annealing step is performed for a time period within in the range of 6 minutes to 7 minutes. In some embodiments, the Annealing step is performed for a time period within in the range of 7 minutes to 8 minutes. In some embodiments, the Annealing step is performed for a time period within in the range of 8 minutes to 9 minutes. In some embodiments, the Annealing step is performed for a time period within in the range of 9 minutes to 10 minutes.

In some embodiments, the Annealing step is performed for 1 minute. In some embodiments, the Annealing step is performed for less than one minute. In some embodiments, the Annealing step is performed for 2 minutes. In some embodiments, the Annealing step is performed for 3 minutes. In some embodiments, the Annealing step is performed for 4 minutes. In some embodiments, the Annealing step is performed for 5 minutes. In some embodiments, the Annealing step is performed for 6 minutes. In some embodiments, the Annealing step is performed for 7 minutes. In some embodiments, the Annealing step is performed for 8 minutes. In some embodiments, the Annealing step is performed for 9 minutes. In some embodiments, the Annealing step is performed for 10 minutes. In some embodiments, the Annealing step is performed for less than 10 minutes. In some embodiments, the Annealing step is performed for greater than 10 minutes. In some embodiments, the Annealing step is performed for greater than 10 seconds.

In some embodiments, the Elongation step is performed for a time period selected from the range consisting of: 30 seconds to 1 minute. In some embodiments, the Elongation step is performed for a time period selected from the range consisting of: 1 minute to 2 minutes. In some embodiments, the Elongation step is performed for a time period selected from the range consisting of: 2 minutes to 3 minutes. In some embodiments, the Elongation step is performed for a time period selected from the range consisting of: 3 minutes to 4 minutes. In some embodiments, the Elongation step is performed for a time period selected from the range consisting of: 4 minutes to 5 minutes. In some embodiments, the Elongation step is performed for a time period selected from the range consisting of: 5 minutes to 6 minutes. In some embodiments, the Elongation step is performed for a time period selected from the range consisting of: 6 minutes to 7 minutes. In some embodiments, the Elongation step is performed for a time period selected from the range consisting of: 7 minutes to 8 minutes. In some embodiments, the Elongation step is performed for a time period selected from the range consisting of: 8 minutes to 9 minutes. In some embodiments, the Elongation step is performed for a time period selected from the range consisting of: 9 minutes to 10 minutes. In some embodiments, the Elongation step is performed for a time period selected from the range consisting of: 10 minutes to 11 minutes. In some embodiments, the Elongation step is performed for a time period selected from the range consisting of: 11 minutes to 12 minutes. In some embodiments, the Elongation step is performed for a time period selected from the range consisting of: 12 minutes to 13 minutes. In some embodiments, the Elongation step is performed for a time period selected from the range consisting of: 13 minutes to 14 minutes. In some embodiments, the Elongation step is performed for a time period selected from the range consisting of: 14 minutes to 15 minutes. In some embodiments, the Elongation step is performed for a time period selected from the range consisting of: 15 minutes to 16 minutes. In some embodiments, the Elongation step is performed for a time period selected from the range consisting of: 16 minutes to 17 minutes. In some embodiments, the Elongation step is performed for a time period selected from the range consisting of: 17 minutes to 18 minutes. In some embodiments, the Elongation step is performed for a time period selected from the range consisting of: 18 minutes to 19 minutes. In some embodiments, the Elongation step is performed for a time period selected from the range consisting of: 19 minutes to 20 minutes. In some embodiments, the Elongation step is performed for a time period selected from the range consisting of: 20 minutes to 21 minutes. In some embodiments, the Elongation step is performed for a time period selected from the range consisting of: 21 minutes to 22 minutes. In some embodiments, the Elongation step is performed for a time period selected from the range consisting of: 22 minutes to 23 minutes. In some embodiments, the Elongation step is performed for a time period selected from the range consisting of: 23 minutes to 24 minutes. In some embodiments, the Elongation step is performed for a time period selected from the range consisting of: 24 minutes to 25 minutes. In some embodiments, the Elongation step is performed for a time period selected from the range consisting of: 25 minutes to 26 minutes. In some embodiments, the Elongation step is performed for a time period selected from the range consisting of: 26 minutes to 27 minutes. In some embodiments, the Elongation step is performed for a time period selected from the range consisting of: 27 minutes to 28 minutes. In some embodiments, the Elongation step is performed for a time period selected from the range consisting of: 28 minutes to 29 minutes. In some embodiments, the Elongation step is performed for a time period selected from the range consisting of: 29 minutes to 30 minutes. In some embodiments, the Elongation step is performed for a time period selected from the range consisting of: 30 minutes to 31 minutes. In some embodiments, the Elongation step is performed for less than 30 seconds. In some embodiments, the Elongation step is performed for greater than 30 minutes.

In some embodiments, the Final Elongation step is not performed. In some embodiments, the Final Elongation step is performed for less than 30 seconds. In some embodiments, the Final Elongation step is performed for a time period selected from the range consisting of: 30 seconds to 1 minute. In some embodiments, the Final Elongation step is performed for a time period selected from the range consisting of: 1 minute to 2 minutes. In some embodiments, the Final Elongation step is performed for a time period selected from the range consisting of: 2 minutes to 3 minutes. In some embodiments, the Final Elongation step is performed for a time period selected from the range consisting of: 3 minutes to 4 minutes. In some embodiments, the Final Elongation step is performed for a time period selected from the range consisting of: 4 minutes to 5 minutes. In some embodiments, the Final Elongation step is performed for a time period selected from the range consisting of: 5 minutes to 6 minutes. In some embodiments, the Final Elongation step is performed for a time period selected from the range consisting of: 6 minutes to 7 minutes. In some embodiments, the Final Elongation step is performed for a time period selected from the range consisting of: 7 minutes to 8 minutes. In some embodiments, the Final Elongation step is performed for a time period selected from the range consisting of: 8 minutes to 9 minutes. In some embodiments, the Final Elongation step is performed for a time period selected from the range consisting of: 9 minutes to 10 minutes. In some embodiments, the Final Elongation step is performed for a time period selected from the range consisting of: 10 minutes to 11 minutes. In some embodiments, the Final Elongation step is performed for a time period selected from the range consisting of: 11 minutes to 12 minutes. In some embodiments, the Final Elongation step is performed for a time period selected from the range consisting of: 12 minutes to 13 minutes. In some embodiments, the Final Elongation step is performed for a time period selected from the range consisting of: 13 minutes to 14 minutes. In some embodiments, the Final Elongation step is performed for a time period selected from the range consisting of: 14 minutes to 15 minutes. In some embodiments, the Final Elongation step is performed for a time period selected from the range consisting of: 15 minutes to 16 minutes. In some embodiments, the Final Elongation step is performed for a time period selected from the range consisting of: 16 minutes to 17 minutes. In some embodiments, the Final Elongation step is performed for a time period selected from the range consisting of: 17 minutes to 18 minutes. In some embodiments, the Final Elongation step is performed for a time period selected from the range consisting of: 18 minutes to 19 minutes. In some embodiments, the Final Elongation step is performed for a time period selected from the range consisting of: 19 minutes to 20 minutes. In some embodiments, the Final Elongation step is performed for a time period selected from the range consisting of: 20 minutes to 21 minutes. In some embodiments, the Final Elongation step is performed for a time period selected from the range consisting of: 21 minutes to 22 minutes. In some embodiments, the Final Elongation step is performed for a time period selected from the range consisting of: 22 minutes to 23 minutes. In some embodiments, the Final Elongation step is performed for a time period selected from the range consisting of: 23 minutes to 24 minutes. In some embodiments, the Final Elongation step is performed for a time period selected from the range consisting of: 24 minutes to 25 minutes. In some embodiments, the Final Elongation step is performed for a time period selected from the range consisting of: 25 minutes to 26 minutes. In some embodiments, the Final Elongation step is performed for a time period selected from the range consisting of: 26 minutes to 27 minutes. In some embodiments, the Final Elongation step is performed for a time period selected from the range consisting of: 27 minutes to 28 minutes. In some embodiments, the Final Elongation step is performed for a time period selected from the range consisting of: 28 minutes to 29 minutes. In some embodiments, the Final Elongation step is performed for a time period selected from the range consisting of: 29 minutes to 30 minutes. In some embodiments, the Final Elongation step is performed for a time period selected from the range consisting of: 30 minutes to 31 minutes. In some embodiments, the Final Elongation step is performed for greater than 30 minutes.

In some embodiments, movement of the reaction vessel between the temperature-controlled baths corresponds to the reaction. In some embodiments, the PCR product produced by the method is in batch sizes selected from the range from 1 mL to 5 L. In some embodiments, the batch size was less than 1 mL. In some embodiments, the batch size is greater than 5 L. In some embodiments, the batch size is within a range selected from the group consisting of: 1 mL to 100 mL, 50 mL to 200 mL, 150 mL to 300 mL, 250 mL to 400 mL, 350 mL to 500 mL, 450 mL to 600 mL, 550 mL to 700 mL, 650 mL to 800 mL, 750 mL to 900 mL, 850 mL to 1L, 950 mL to 1.5 L, 1 L to 2 L, 1.5 L to 3 L, 2 L to 3.5 L, 2.5 L to 4 L, 3 L to 4.5 L, and 3.5 L to 5 L.

### II. System

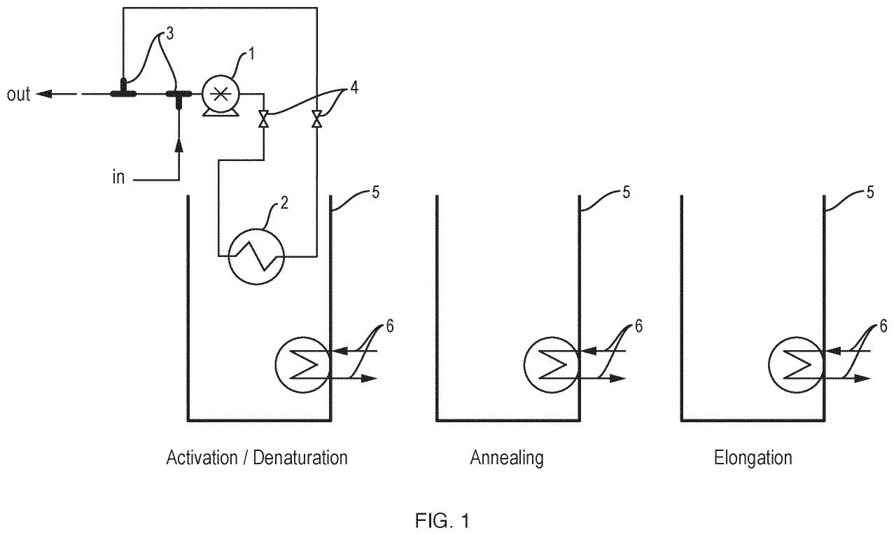

Some embodiments herein describe a system for large-scale production of nucleic acids using polymerase chain reaction (PCR). Some embodiments include the system shown in FIG. 1.

In some embodiments, the system is capable of rapid heat transfers due to the high surface-area-to-volume-ratio of the reaction vessel 2 and the physical movement of the bioreactor between the temperature-controlled baths 5 by the manipulator. In some embodiments, turbulence is introduced within the reaction vessel 2 by incorporation of a pump 1 for circulation of the reaction mixture within capillary tubing 2 and by choice of capillary winding designs.

In some embodiments, the system described herein exploits the versatility of a robotic element as a manipulator to physically move the reaction vessel 2. In some embodiments, any PCR time protocol may be programmed by software modification for multiple PCR protocols to circumvent limitations of all previously developed continuous capillary PCR reactors where the reaction time is fully dependent on the capillary tubing 2 dimensions. Merger of enhanced heat and mass transfers in capillary tubing 2 with a physically moveable batch between multiple heated baths not only meets the stringent parameter control required for PCRs, but also provides a reliable path to flexible, scalable production of nucleic acids.

### a. Capillary Tubing

Some embodiments of the method described herein are performed using a system comprising capillary tubing 2 as a reaction vessel. In some embodiments, the flow rate and type of winding influences secondary flows (Dean Vortices) within the capillary tubing 2 leading to enhanced heat and mass transfer. In some embodiments, the flow rate and type of winding influences these secondary flow (Dean vortices) within the capillary tubing 2 to avoid hotspots. Overall, these characteristics improve the quality and reproducibility of the PCR product.

In some embodiments, the capillary tubing 2 is wound on frames and submerged within the temperature-controlled bath 5. The frame may be constructed in any design and made of any material capable of supporting the type of winding and weight of the filled capillary tubing 2. In some embodiments, the capillary tubing 2 may be wound onto the frame in any design which encourages turbulent mixing of the reagents and also allows optimum contact between the thermal medium and outer surface of the capillary tubing 2. In some embodiments, capillary tubing 2 is wound in a design selected from the list consisting of: simple helical coils, 180 degree turns, and other more complex coiled flow inverters (CFI). (See, A. K. Saxena and K. D. P. Nigam, AICHE J., 1984, 30, 363-368, which is hereby incorporated by reference in its entirety)

In some embodiments, the reaction vessel is capillary tubing 2 comprising any suitable material. In some embodiments, the capillary tubing 2 comprises at least one material selected from the group consisting of: metals, plastics, and silicones. In some embodiments, the capillary tubing 2 comprises at least one metal. In some embodiments, the capillary tubing 2 is made of a metal. In some embodiments, the capillary tubing 2 comprises a metal selected from the group consisting of: stainless steel, copper, and hastelloy. In some embodiments, the capillary tubing 2 comprises stainless steel. In some embodiments, the capillary tubing 2 comprises copper. In some embodiments, the capillary tubing 2 comprises hastelloy. In some embodiments, the capillary tubing 2 comprises at least one plastic selected from the group consisting of: perfluoroalkoxy (PFA), polysulfone, fluorinated ethylene propylene (FEP), and polyethylene (PE). In some embodiments, the capillary tubing 2 comprises PFA. In some embodiments, the capillary tubing 2 comprises polysulfone. In some embodiments, the capillary tubing 2 comprises FEP. In some embodiments, the capillary tubing 2 comprises PE.

In some embodiments, the capillary tubing 2 is sterilizable. In some embodiments, the capillary tubing 2 is single use. In some embodiments, the capillary tubing 2 is silicone tubing. In some embodiments, the capillary tubing 2 is platinum-cured silicone tubing. In some embodiments, the capillary tubing 2 is peroxide-cured silicone tubing. In some embodiments, the capillary tubing 2 is sterilizable and single use platinum cured silicone tubing.

In some embodiments, the internal diameter of the capillary tubing 2 is between 0.5 mm to 10 mm. In some embodiments, the internal diameter is within a range select from the group consisting of: 0.5 to 1.5 mm, 1.0 mm to 2.0 mm, 1.5 mm to 2.5 mm, 2.0 mm to 3.0 mm, 2.5 mm to 3.5 mm, 3.0 mm to 4.0 mm, 3.5 mm to 4.5 mm, 4.0 mm to 5.0 mm, 4.5 mm to 5.5 mm, 5.0 mm to 6.0 mm, 5.5 mm to 7.0 mm, 6.0 mm to 7.0 mm, 6.5 mm to 7.5 mm, 7.0 mm to 8.0 mm, 7.5 mm to 8.5 mm, 8.0 mm to 9.0 mm, 8.5 mm to 9.5 mm, and 9.0 mm to 10.0 mm. In some embodiments, the internal diameter of the capillary tubing 2 is 0.5 mm. In some embodiments, the internal diameter of the capillary tubing 2 is 0.6 mm. In some embodiments, the internal diameter of the capillary tubing 2 is 0.7 mm. In some embodiments, the internal diameter of the capillary tubing 2 is 0.8 mm. In some embodiments, the internal diameter of the capillary tubing 2 is 0.9 mm. In some embodiments, the internal diameter of the capillary tubing 2 is 1.0 mm. In some embodiments, the internal diameter of the capillary tubing 2 is 1.1 mm. In some embodiments, the internal diameter of the capillary tubing 2 is 1.2 mm. In some embodiments, the internal diameter of the capillary tubing 2 is 1.3 mm. In some embodiments, the internal diameter of the capillary tubing 2 is 1.4 mm. In some embodiments, the internal diameter of the capillary tubing 2 is 1.5 mm. In some embodiments, the internal diameter of the capillary tubing 2 is 1.6 mm. In some embodiments, the internal diameter of the capillary tubing 2 is 1.7 mm. In some embodiments, the internal diameter of the capillary tubing 2 is 1.8 mm. In some embodiments, the internal diameter of the capillary tubing 2 is 1.9 mm. In some embodiments, the internal diameter of the capillary tubing 2 is 2.0 mm. In some embodiments, the internal diameter of the capillary tubing 2 is 2.1 mm. In some embodiments, the internal diameter of the capillary tubing 2 is 2.2 mm. In some embodiments, the internal diameter of the capillary tubing 2 is 2.3 mm. In some embodiments, the internal diameter of the capillary tubing 2 is 2.4 mm. In some embodiments, the internal diameter of the capillary tubing 2 is 2.5 mm. In some embodiments, the internal diameter of the capillary tubing 2 is 2.6 mm. In some embodiments, the internal diameter of the capillary tubing 2 is 2.7 mm. In some embodiments, the internal diameter of the capillary tubing 2 is 2.8 mm. In some embodiments, the internal diameter of the capillary tubing 2 is 2.9 mm. In some embodiments, the internal diameter of the capillary tubing 2 is 3.0 mm. In some embodiments, the internal diameter of the capillary tubing 2 is 3.1 mm. In some embodiments, the internal diameter of the capillary tubing 2 is 3.2 mm. In some embodiments, the internal diameter of the capillary tubing 2 is 3.3 mm. In some embodiments, the internal diameter of the capillary tubing 2 is 3.4 mm. In some embodiments, the internal diameter of the capillary tubing 2 is 3.5 mm. In some embodiments, the internal diameter of the capillary tubing 2 is 3.6 mm. In some embodiments, the internal diameter of the capillary tubing 2 is 3.7 mm. In some embodiments, the internal diameter of the capillary tubing 2 is 3.8 mm. In some embodiments, the internal diameter of the capillary tubing 2 is 3.9 mm. In some embodiments, the internal diameter of the capillary tubing 2 is 4.0 mm. In some embodiments, the internal diameter of the capillary tubing 2 is 4.1 mm. In some embodiments, the internal diameter of the capillary tubing 2 is 4.2 mm. In some embodiments, the internal diameter of the capillary tubing 2 is 4.3 mm. In some embodiments, the internal diameter of the capillary tubing 2 is 4.4 mm. In some embodiments, the internal diameter of the capillary tubing 2 is 4.5 mm. In some embodiments, the internal diameter of the capillary tubing 2 is 4.6 mm. In some embodiments, the internal diameter of the capillary tubing 2 is 4.7 mm. In some embodiments, the internal diameter of the capillary tubing 2 is 4.8 mm. In some embodiments, the internal diameter of the capillary tubing 2 is 4.9 mm. In some embodiments, the internal diameter of the capillary tubing 2 is 5.0 mm. In some embodiments, the internal diameter of the capillary tubing 2 is 5.1 mm. In some embodiments, the internal diameter of the capillary tubing 2 is 5.2 mm. In some embodiments, the internal diameter of the capillary tubing 2 is 5.3 mm. In some embodiments, the internal diameter of the capillary tubing 2 is 5.4 mm. In some embodiments, the internal diameter of the capillary tubing 2 is 5.5 mm. In some embodiments, the internal diameter of the capillary tubing 2 is 5.6 mm. In some embodiments, the internal diameter of the capillary tubing 2 is 5.7 mm. In some embodiments, the internal diameter of the capillary tubing 2 is 5.8 mm. In some embodiments, the internal diameter of the capillary tubing 2 is 5.9 mm. In some embodiments, the internal diameter of the capillary tubing 2 is 6.0 mm. In some embodiments, the internal diameter of the capillary tubing 2 is 6.1 mm. In some embodiments, the internal diameter of the capillary tubing 2 is 6.2 mm. In some embodiments, the internal diameter of the capillary tubing 2 is 6.3 mm. In some embodiments, the internal diameter of the capillary tubing 2 is 6.4 mm. In some embodiments, the internal diameter of the capillary tubing 2 is 6.5 mm. In some embodiments, the internal diameter of the capillary tubing 2 is 6.6 mm. In some embodiments, the internal diameter of the capillary tubing 2 is 6.7 mm. In some embodiments, the internal diameter of the capillary tubing 2 is 6.8 mm. In some embodiments, the internal diameter of the capillary tubing 2 is 6.9 mm. In some embodiments, the internal diameter of the capillary tubing 2 is 7.0 mm. In some embodiments, the internal diameter of the capillary tubing 2 is 7.1 mm. In some embodiments, the internal diameter of the capillary tubing 2 is 7.2 mm. In some embodiments, the internal diameter of the capillary tubing 2 is 7.3 mm. In some embodiments, the internal diameter of the capillary tubing 2 is 7.4 mm. In some embodiments, the internal diameter of the capillary tubing 2 is 7.5 mm. In some embodiments, the internal diameter of the capillary tubing 2 is 7.6 mm. In some embodiments, the internal diameter of the capillary tubing 2 is 7.7 mm. In some embodiments, the internal diameter of the capillary tubing 2 is 7.8 mm. In some embodiments, the internal diameter of the capillary tubing 2 is 7.9 mm. In some embodiments, the internal diameter of the capillary tubing 2 is 8.0 mm. In some embodiments, the internal diameter of the capillary tubing 2 is 8.1 mm. In some embodiments, the internal diameter of the capillary tubing 2 is 8.2 mm. In some embodiments, the internal diameter of the capillary tubing 2 is 8.3 mm. In some embodiments, the internal diameter of the capillary tubing 2 is 8.4 mm. In some embodiments, the internal diameter of the capillary tubing 2 is 8.5 mm. In some embodiments, the internal diameter of the capillary tubing 2 is 8.6 mm. In some embodiments, the internal diameter of the capillary tubing 2 is 8.7 mm. In some embodiments, the internal diameter of the capillary tubing 2 is 8.8 mm. In some embodiments, the internal diameter of the capillary tubing 2 is 8.9 mm. In some embodiments, the internal diameter of the capillary tubing 2 is 9.0 mm. In some embodiments, the internal diameter of the capillary tubing 2 is 9.1 mm. In some embodiments, the internal diameter of the capillary tubing 2 is 9.2 mm. In some embodiments, the internal diameter of the capillary tubing 2 is 9.3 mm. In some embodiments, the internal diameter of the capillary tubing 2 is 9.4 mm. In some embodiments, the internal diameter of the capillary tubing 2 is 9.5 mm. In some embodiments, the internal diameter of the capillary tubing 2 is 9.6 mm. In some embodiments, the internal diameter of the capillary tubing 2 is 9.7 mm. In some embodiments, the internal diameter of the capillary tubing 2 is 9.8 mm. In some embodiments, the internal diameter of the capillary tubing 2 is 9.9 mm. In some embodiments, the internal diameter of the capillary tubing 2 is 10.0 mm. In some embodiments, the diameter and length is calculated based on the volume of the batch required, as well as the rate of heat transfer required during the PCR protocol.

In some embodiments, the capillary tubing 2 can be completely or only partially filled with reaction mixture. In some embodiments, the capillary tubing 2 can be completely filled with reaction mixture. In some embodiments, the capillary tubing 2 can be partially filled with reaction mixture. Alternatively, in some embodiments, the reaction mixture can be introduced into the capillary tubing 2 in segmented form. In some embodiments, the reaction mixture is segmented by intercalation of an non-miscible gas, liquid, or oil forming sequential and separate segments.

In some embodiments, a connector 3 creates an inlet into the reaction vessel 2 for the reaction mixture. In some embodiments a connector 3 creates an outlet from the reaction vessel 2 for the reaction mixture.

In some embodiments, at least one of the connectors 3 or 4 is selected from the group consisting of: an automated three-way connector or valve, an automated four-way connector or valve, a manual three-way connector or valve, and a manual four-way connector or valve, an automated union connector, a manual union connector. In some embodiments, at least one connector 3 or 4 is an automated union connector. In some embodiments, at least one connector 3 or 4 is an automated three-way connector. In some embodiments, at least one connector 3 or 4 is an automated four-way connector. In some embodiments, at least one connector 3 or 4 is a manual union connector. In some embodiments, at least one connector 3 or 4 is a manual three-way connector. In some embodiments, at least one connector 3 or 4 is a manual four-way connector.

### b. Circulation of the Reaction Mixture

In some embodiments, the reaction mixture may remain in a static environment. In some embodiments, the reaction mixture is mixed within the reaction vessel 2. In some embodiments, mixing and movement of the reaction mixture within the reactor vessel 2 can be accomplished using a circulation device 1. In some embodiments, the circulation device 1 is a pump or an oscillatory flow.

In some embodiments, the flow rate of the reaction mixture is independent of reaction time for the steps of the PCR protocol. In some embodiments, the flow rate of the reaction mixture is determined from at least one characteristic selected from the group consisting of: the optimal amount of mixing and turbulence, the dimensions of the capillary tubing 2, and the winding design of the capillary tubing 2. In some embodiments, the flow rate of the reaction mixture is determined from the optimal amount of mixing and turbulence. In some embodiments, the flow rate of the reaction mixture is determined from the dimensions of the capillary tubing 2. In some embodiments, the flow rate of the reaction mixture is determined from the winding design of the capillary tubing 2.

In some embodiments, filling, emptying, and/or movement of the reaction mixture through the reaction vessel can be performed with any type of pump 1. In some embodiments, the pump 1 is a pump selected from the group consisting of: a peristaltic pump, a gear pump, a lobe pump, a membrane pump, an impeller pump, a diaphragm pump and a syringe pump. In some embodiments, at least one pump is a peristaltic pump. For example, the peristaltic pump may be easily disposable with sterilizable tubing. In some embodiments, the peristaltic pump is a easily cleanable, such as by wiping it down. In some embodiments, at least one pump 1 is a gear pump. In some embodiments, at least one pump is a lobe pump. In some embodiments, at least one pump is a membrane pump. In some embodiments, at least one pump 1 is a syringe pump. In some embodiments, a pump 1 is mounted on the robotic arm. For example, in some embodiments, the pump 1 is mounted on a robotic arm as shown in FIG. 2.

### cd. Temperature-Controlled Bath

In some embodiments, the temperature-controlled bath 5 may use gas (e. g air or nitrogen) as the thermal medium, which is circulated within the temperature-controlled bath 5, ensuring a uniform temperature profile by convection. In some embodiments, the temperature in each bath is separately controlled using a heating element 6 for electrical heating. In some embodiments, the temperature in each bath is separately controlled with a thermostat. In some embodiments, any thermal fluid having suitable chemical compatibility with the reaction vessel 2 may be used as the thermal medium. In some embodiments, water is the thermal medium.

In some embodiments, the heated fluid baths are covered with lids. In some embodiments, the lids are formed lids. In some embodiments, the lids act as drip trays. In some embodiments, the lids contain an outlet for removal of collected fluid. In some embodiments, the lid of each temperature-controlled bath 5 contains sealed ports for entry and exit of capillary tubing 2. In some embodiments, connectors 4 between the capillary tubing and pumps are located outside of the baths to avoid contamination of the PCR product by the fluid or air in the bath. In some embodiments, the opening and closing of the lids is performed by a vacuum lifter in combination with a manipulator.

Any type of suitable connections may be used. In some embodiments, at least one of the connectors 4 are a union connector.

In some embodiments, the number of temperature-controlled baths 5 is dependent on the complexity of the PCR protocol. In some embodiments, the number of temperature-controlled baths 5 is selected from the group consisting of: at least 1, at least 2, at least 3, at least 4, at least 5, and at least 6. In some embodiments, the number of temperature-controlled baths 5 is within the range of 2 to 6. In some embodiments, the number of temperature-controlled baths 5 is 2. In some embodiments, the number of temperature-controlled baths 5 is 3. In some embodiments, the Activation and Denaturation steps may occur in the same temperature-controlled baths 5. In some embodiments, the Activation and Denaturation steps may occur in the same temperature-controlled baths. In some embodiments, the Activation, Denaturation, Annealing, Elongation, and Final Elongation steps each occur in separate temperature-controlled baths 5. In some embodiments, more than one PCR step is performed in each temperature-controlled bath 5. In some embodiments, each PCR step is performed in a different temperature-controlled bath 5.

In some embodiments, the temperature of each temperature-controlled bath 5 is determined by the template, primer, and polymerase used. In some embodiments, the temperature for the Activation and Denaturation steps is within the range of 85° C. to 100° C. In some embodiments, the temperature for the Annealing step is within the range of 55° C. to 75° C. In some embodiments, the temperature for the Elongation step or the Final Elongation step is within the range of 65° C. to 80° C.

### d. Mobility of Reaction Vessel

In some embodiments, movement of the reaction vessel 2 between the temperature-controlled baths 5 can be accomplished with any suitable manipulator. In some embodiments, the manipulator is a multi-axis pulley or a robot. In some embodiments, the manipulator is a multi-axis pulley. In some embodiments, 2-5 manipulators are used. In some embodiments, the manipulators are x, y linear in movement. In some embodiments, three manipulators are used. In some embodiments, the manipulator controls movement of the capillary package. In some embodiments, the manipulator controls movement of a drip tray, which follows the movement of the capillary package, to collect droplets between the temperature-controlled baths.

In some embodiments, the manipulator is a robot. In some embodiments, movement of the manipulator is automatically controlled. In some embodiments, movement of the manipulator is programmable. In some embodiments, movement of the manipulator is reprogrammable. In some embodiments, movement of the manipulator is automatically controlled. In some embodiments, the manipulator move the reaction vessel in more than two directions. In some embodiments, the manipulators move the reaction vessel in x, y, and z axis.

In some embodiments, the manipulator is operated manually using a control panel. In some embodiments, the manipulator is remotely controlled using a pendant or a remote. In some embodiments, the manipulator is integrated into a process control automation software. In some embodiments, the manipulator is a robotic arm fixed in place. In some embodiments, the robotic arm is moveable in three or more axes. In some embodiments, the position of the robotic arm is not limited. For example, the robotic arm is mounted overhead, behind, in-front of, or in-line with the temperature-controlled baths in some embodiments. In some embodiments, the robotic arm is mounted overhead of the temperature-controlled baths 5. In some embodiments, the robotic arm is mounted behind the temperature-controlled baths. In some embodiments, the robotic arm is mounted in front of the temperature-controlled baths 5. In some embodiments, the robotic arm is mounted in-line with the temperature-controlled baths.

FIG. 1 shows an embodiment of a robotic arm featuring rotary joints on the arm connected to a twisting joint at the base. In some embodiments, these six movable axes provide freedom of movement in the x, y, and z directions.

### e. Prevention of Cross-Contamination

Avoiding cross-contamination is important when using fluid filled tanks in a controlled Good Manufacturing Process (GMP) production cleanroom. Common cross-contamination risks in upscaling PCR include: open handling of temperature-controlled baths 5 and moving parts within the controlled GMP cleanroom. In some embodiments, the movable parts includes drip trays, multiple manipulators, aspirators, and other parts in contact with the thermal fluid. In some embodiments, the risk of cross-contamination is reduced by separating other components of the system from the thermal fluid and fully cleanable movable parts.

In some embodiments, the system comprises a separated technical area on the back containing all supporting equipment such as electrical cables, heated elements, moveable linear units, and the control panel. In some embodiments, the separated technical area is never in contact with the reaction mixture and the risk of cross contamination is eliminated. In some embodiments, an enclosed, ventilated working area at the front comprising 2-5 areas, of which at least two are temperature-controlled baths 5.

In some embodiments, the temperature-controlled bath 5 are designed according to hygienic standards, such as minimizing angled edges and avoiding hold-up or dead volumes. In some embodiments, the temperature-controlled baths 5 are designed to be completely emptied and cleaned.

In some embodiments, all parts wetted by the thermal fluid wetted of the temperature-controlled bath 5 including piping, pump heads, and heat exchangers are resistant to the thermal fluid at temperatures of at least to 98° C. In some embodiments, all parts wetted by the thermal fluid wetted of the temperature-controlled bath 5 including piping, pump heads, and heat exchangers are compatible with cleaning protocols typical of biopharmaceutical production.

In some embodiments, one of the areas can serve as a load/unload docking station, or alternatively, as a cooled fluid filled bath to quickly quench the process after the final elongation.

In some embodiments, at least one drip tray connected to a clean room aspirator is positioned between temperature-controlled baths 5 and is used to remove droplets during movement of the capillary package.

In some embodiments, the moving parts of the manipulators are housed in the rear technical area to reduce the risk of cross-contamination from fluid or vapor. In some embodiments, Within the working area, the manipulators are placed behind faceplates or covers suitable for cleanrooms.

In some embodiments, the system is automated and controlled through software compliant with FDA 21 CFR Part 11 to provide an audit trail for all changes.

### III. Definitions

Unless defined otherwise, all technical and scientific terms used herein have the same meaning as commonly understood by one of ordinary skill in the art to which this invention pertains.

As used herein, the singular forms “a”, “an,” and “the” include plural unless the context clearly dictates otherwise.

As used herein, the term “capillary package” refers to the combination of a reaction vessel wound onto a frame and peristaltic pump fitted with a pump head which can be easily disassembled and cleaned, and a substitute lid ensuring closure of the temperature-controlled bath currently in use.

As used herein, the term “master mix” refers to a solution including the components required for a PCR reaction, for example, a polymerase, at least one primer, and deoxynucleoside triphosphates (dNTPs).

As used herein, the term “reaction mixture” refers to a master mix combined with the nucleic acid template.

As used herein, the term “thermal transfer” refers to heating a mixture, such as the reaction mixture herein, by contact with a fluid to bring the mixture to the temperature of the fluid.

### EXAMPLES

### Example 1. Pcr Protocol

The following is a working example of a PCR protocol for target DNA of 2079 base pairs using the newly designed reaction vessel as described in some embodiments described herein. The master mix was prepared in a 50 mL Eppendorf conical tube as follows: 3 mL 5× Reaction Buffer; 7.8 mL RNA Water; 3 mL 5× Enhancer appropriate for the polymerase; 300 μL dNTP (10 mM); 75 μL Primer (Forward Primer 100 μM); 75 μL Primer (Reverse Primer 100 μM); and 150 μL DNA Polymerase (2000 U/ml). The master mix was combined with the 600 μL Template (100 pg/μL) to create the reaction mixture.

The reaction vessel was formed by preparing 7 m of 1.58 mm internal diameter (ID) and 0.8 mm WT of platinum-cured silicone tubing in helical coils. Three different temperatures were used for the different temperature-controlled baths. As Activation and Denaturation are performed at the same temperature, one temperature-controlled bath was used for both reaction steps at a temperature of 98° C. The Annealing step was performed at 63° C. The Elongation step was performed at 72° C.

The following PCR protocol was programmed into the robotic automation software: Activation step—112 seconds; 30 cycles of Denaturation—37 seconds, Annealing—53 seconds, Elongation—66 seconds; and Final Elongation—120 seconds.

To begin the reaction, the pump was started and the reaction mixture (14 mL) was transferred from the Eppendorf conical tube to the reaction vessel using a peristaltic pump. The pump flow rate was set to 15 mL/minute. The reaction mixture was continuously fed through the reaction vessel by the pumping action. The PCR product was transferred into a new sterile conical tube by the peristaltic pump. A 2 μL sample of the product was diluted to 20 μL (10×) and analyzed on 1% Agarose Gel using electrophoresis. A 2 μL sample of the product was collected and diluted to 20 μL (10×), and electrophoresis was performed on a 1% agarose gel to confirm the presence of the expected 2079 base pair (bp) product.

### EQUIVALENTS

All ranges for formulations recited herein include ranges therebetween and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4, or 3.1 or more.

Reference throughout this specification to “one embodiment,” “certain embodiments,” “one or more embodiments,” “some embodiments,” or “an embodiment” indicates that a feature, structure, material, or characteristic described is included some embodiments of the disclosure. Therefore, the appearances of the phrases such as “in one or more embodiments,” “in certain embodiments,” “in one embodiment,” “some embodiments,” or “in an embodiment” throughout this specification are not necessarily referring to the same embodiment.

Publications of patent applications and patents and other non-patent references, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A system for performing polymerase chain reaction (PCR) comprising:
a) a reaction vessel comprising capillary tubing wound around a frame;
b) a circulation device for circulating a reaction mixture through the reaction vessel;
c) at least two temperature-controlled baths holding a fluid for thermal transfer to the reaction mixture; and
d) at least one manipulator to transfer the reaction vessel among the temperature-controlled baths while performing PCR.

2. The system of claim 1, wherein the manipulator is a multi-axis pulley or a robotic arm.

3. The system of claim 2, wherein the manipulator is a robotic arm.

4. The system of claim 3, wherein the robotic arm is automated.

5. The system of claim 2, wherein the robotic arm is programmable.

6. The system of claim 3, wherein the robotic arm is manual.

7. The system of claim 1, wherein the robotic arm comprises rotary joints.

8. The system of claim 1, wherein the circulation device is a pump.

9. The system of claim 8, the pump is at least one type of pump selected from the group consisting of: a peristaltic pump, a gear pump, a lobe pump, a membrane pump, and a syringe pump.

10. The system of claim 1, wherein the system is a discrete batch bioreactor.

11. The system of claim 1, wherein the capillary tubing is wound around the frame in a design selected from the designs consisting of: simple helical coils, 180 degree turns, and other more complex coiled flow inverters (CFI).

12. The system of claim 1, wherein the capillary tubing comprises at least one material selected from the group consisting of: metals, plastics, and silicones.

13. The system of claim 12, wherein the capillary tubing is silicone tubing.

14. The system of claim 13, wherein the capillary tubing is platinum-cured silicone tubing.

15. The system of claim 14, wherein the capillary tubing is peroxide-cured silicone tubing.

16. The system of claim 1, wherein the capillary tubing is sterilizable.

17. The system of claim 1, wherein the capillary tubing is single use.

18. The system of claim 1, wherein the circulation device creates oscillatory flow.

19. The system of claim 1, wherein the system comprises three temperature-controlled baths.

20. A method for performing polymerase chain reaction (PCR) using the system of claim 1 to produce a product, the method comprising:
a) filling the reaction vessel with the reaction mixture;
b) introducing turbulence using the circulation device to circulate the reaction mixture;
c) submerging the reaction vessel into a first temperature-controlled bath corresponding to reaction conditions for a denaturation step of PCR; and
d) transferring the reaction vessel using a manipulator from the first temperature-controlled bath to a second temperature-controlled bath corresponding to reaction conditions for a different step of PCR.

21. The method of claim 20, wherein the product is produced in a milliliter or a liter volume scale.

22. The method of claim 20, wherein each submerging step corresponds to a step of PCR.

23. The method of claim 20, further comprising programming the movement of the robotic arm prior to performing PCR.

24. The method of claim_20, wherein the product is produced in a batch size from 1 mL to 5 L.

25. The method of claim 24, wherein the product is produced in a batch size within a range selected from the group consisting of: 1 mL to 100 mL, 50 mL to 200 mL, 150 mL to 300 mL, 250 mL to 400 mL, 350 mL to 500 mL, 450 mL to 600 mL, 550 mL to 700 mL, 650 mL to 800 mL, 750 mL to 900 mL, 850 mL to 1L, 950 mL to 1.5 L, 1 L to 2 L, 1.5 L to 3 L, 2 L to 3.5 L, 2.5 L to 4 L, 3 L to 4.5 L, and 3.5 L to 5 L.

26. The method of claim_20, further comprising prior to the filling step calculating the diameter and length of the capillary tubing based on the volume of a batch.

27. The method of any one of claims claim 20, further comprising, prior to the filling step, calculating the diameter and length of the capillary tubing based on the rate of heat transfer required for PCR.

28. The method of claim 20, further comprising filling the reaction vessel with the reaction mixture in a segmented form.

29. The method of claim 20, further comprising filling the reaction vessel with the reaction mixture in a non-segmented form.

30. The method of claim 20, further comprising, prior to introducing turbulence, calculating the flow rate of the reaction mixture from at least one characteristic selected from the group consisting of: the optimal amount of mixing and turbulence, the dimensions of the capillary tubing, and the winding design of the capillary tubing.
